(19) United States
(12) Patent Application Publication
(10) Pub. No.: US 2024/0157172 A1
(43) Pub. Date: May 16, 2024

(54) HETEROGENEOUS RADIOTHERAPY DOSE PREDICTION USING GENERATIVE ARTIFICIAL INTELLIGENCE

(71) Applicant: SIEMENS HEALTHINEERS INTERNATIONAL AG, STEINHAUSEN (CH)

(72) Inventors: Riqiang GAO, Plainsboro, NJ (US); Bin LOU, Princeton Junction, NJ (US); Ali KAMEN, Skillman, NJ (US)

(73) Assignee: SIEMENS HEALTHINEERS INTERNATIONAL AG STEINHAUSEN, STEINHAUSEN (CH)

(21) Appl. No.: 18/387,736

(22) Filed: Nov. 7, 2023

Related U.S. Application Data
(60) Provisional application No. 63/382,546, filed on Nov. 7, 2022.

(51) Int. Cl. A61N 5/10; G06N 3/08; G16H 20/40

(52) U.S. Cl. CPC A61N 5/1031; G06N 3/08; G16H 20/40; A61N 2005/1041

(57) ABSTRACT

Provided herein are methods and systems for training and executing an AI model to generate a predicted dose map. In an example, a method comprises generating a training dataset comprising at least a medical image, structure mask(s) for structure(s) within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities; training an artificial intelligence model using the training dataset, such that the artificial intelligence model is configured to receive data associated with a new patient and generate a predicted dose map for the new patient indicating dosage received by one or more internal structures of the new patient; executing the artificial intelligence model for the new patient to receive the predicted dose map for the new patient; and outputting the result.

### CROSS-REFERENCE TO RELATED APPLICATIONS

This application claims priority to U.S. Provisional Application No. 63/382,546, filed Nov. 7, 2022, which is incorporated by reference herein in its entirety for all purposes.

### TECHNICAL FIELD

This application relates generally to using artificial intelligence techniques to model and predict radiotherapy dose distribution attributes.

### BACKGROUND

Radiotherapy is one of the most common treatments for cancer patients. Treatment planning is an essential step of radiotherapy. In the treatment planning phase, the prediction of the expected radiation dose distribution to be delivered is an important task that can improve the efficiency and accuracy of treatment planning. Specifically, dose prediction can be used to effectively implement an optimizer computer model (also referred to as the plan optimizer). For instance, the optimizer may ingest the dose predictions and generate a treatment plan accordingly.

Currently, many software solutions use algorithmic methods to calculate a predicted dose distribution for a patient structure, such as a planning target volume (PTV) or an organ at risk (OAR). Advancements in computer science have empowered planners to create intricate radiotherapy plans aimed at minimizing damage to normal tissues (e.g., OARs). However, these methods have also led to increased demands in terms of time and computing resources.

### SUMMARY

For the aforementioned reasons, there is a desire for a system that can rapidly and accurately analyze patient information and provide various dose predictions. The methods and systems discussed herein can be used in a pipeline of automatic radiotherapy treatment planning, commonly referred to as knowledge-based planning. Knowledge-based planning approaches typically consist of two stages, with the initial stage involving the prediction of the desired or mathematically ideal dose distribution for a patient. Presently, intensity-modulated radiotherapy (IMRT) and volumetric-modulated arc therapy (VMAT) stand as the most frequently used plan types. Even within the same planning modality, such as IMRT, different beam geometry configurations can introduce significant dose heterogeneity, despite sharing the same PTV and OARs. Existing automatic dose prediction models predominantly concentrate on a single modality and/or a single beam configuration, considerably restricting their versatility. In contrast, the AI model discussed herein can utilize conditional generative modeling techniques designed for precise and heterogeneous radiotherapy dose prediction.

AI models can be configured to utilize deep learning techniques, such that they can be implemented in knowledge-based planning. In this approach, the AI model may be trained using a collection of clinically approved plans and their corresponding patient data (e.g., medical images). Once trained, the AI model can predict a three-dimensional dose map for a new patient. However, many of the earlier deep learning methods were mostly suitable for simple situations, such as having a fixed planning type or a consistent beam geometry configuration. Such limitations restrict their broader application and their ability to adapt to various clinical scenarios. Using the methods and system discussed herein, a conditional generative AI model can be configured to incorporate additional information associated with planning types and different beam geometries, thereby improving other approaches. When using the AI model discussed herein, during the prediction phase, users may have the flexibility to select a specific planning type and a combination of beam geometry that align with their clinical needs. Moreover, because the AI model discussed herein does not limit itself to a particular modality, it may be configured for hybrid modalities (e.g., when a combination of both modalities is used on a patient to increase the likelihood of treatment success).

In an embodiment, a method comprises generating, by a processor, a training dataset comprising at least a medical image, one or more structure masks for one or more structures within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities; training, by the processor, an artificial intelligence model using the training dataset, such that the artificial intelligence model is configured to receive data associated with a new patient and generate a predicted dose map for the new patient indicating dosage received by one or more internal structures of the new patient; executing, by the processor, the artificial intelligence model for the new patient to receive the predicted dose map for the new patient; and outputting, by the processor, the predicted dose map for the new patient.

Outputting the predicted dose map may comprise displaying, by the processor, the predicted dose map on an electronic device.

Outputting the predicted dose map may comprise generating, by the processor, a fluence map based on the predicted dose map.

Outputting the predicted dose map may comprise transmitting, by the processor, the predicted dose map to a plan optimizer, whereby the plan optimizer generates a treatment plan for the new patient using the predicted dose map.

The method may further comprise comparing, by the processor, the predicted dose map with at least one clinical goal.

The method may further comprise generating, by the processor, a dose-volume histogram based on the predicted dose map.

The artificial intelligence model may receive a modality indicator from the processor before generating the predicted dose map for the new patient.

In another embodiment, a computer system comprises a server comprising a processor and a non-transitory computer-readable medium containing instructions that when executed by the processor causes the processor to generate a training dataset comprising at least a medical image, one or more structure masks for one or more structures within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities; train an artificial intelligence model using the training dataset, such that the artificial intelligence model is configured to receive data associated with a new patient and generate a predicted dose map for the new patient indicating dosage received by one or more internal structures of the new patient; execute the artificial intelligence model for the new patient to receive the predicted dose map for the new patient; and output the predicted dose map for the new patient.

Outputting the predicted dose map may comprise displaying the predicted dose map on an electronic device.

Outputting the predicted dose map may comprise generating a fluence map based on the predicted dose map.

Outputting the predicted dose map may comprise transmitting the predicted dose map to a plan optimizer, whereby the plan optimizer generates a treatment plan for the new patient using the predicted dose map.

The instructions may further cause the processor to compare the predicted dose map with at least one clinical goal.

The instructions further cause the processor to generate a dose-volume histogram based on the predicted dose map.

The artificial intelligence model receives a modality indicator from the processor before generating the predicted dose map for the new patient.

In another embodiment, a computer system comprising a server comprising a processor and a non-transitory computer-readable medium containing instructions that when executed by the processor causes the processor to generate a training dataset comprising at least a medical image, one or more structure masks for one or more structures within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities; train an artificial intelligence model using the training dataset, such that the artificial intelligence model is configured to receive data associated with a new patient and generate a predicted dose map for the new patient indicating dosage received by one or more internal structures of the new patient; execute the artificial intelligence model for the new patient to receive the predicted dose map for the new patient; and output the predicted dose map for the new patient.

Outputting the predicted dose map may comprise displaying the predicted dose map on an electronic device.

Outputting the predicted dose map comprises generating a fluence map based on the predicted dose map.

Outputting the predicted dose map comprises transmitting the predicted dose map to a plan optimizer, whereby the plan optimizer generates a treatment plan for the new patient using the predicted dose map.

The instructions further cause the processor to compare the predicted dose map with at least one clinical goal.

The instructions may further cause the processor to generate a dose-volume histogram based on the predicted dose map.

The artificial intelligence model receives a modality indicator from the processor before generating the predicted dose map for the new patient.

In another embodiment, a computer system comprises an artificial intelligence model; and a server in communication with the artificial intelligence model, the server configured to generate a training dataset comprising at least a medical image, one or more structure masks for one or more structures within the medical image, an indication of radiotherapy treatment modality, and beam geometry associated with a set of patients, wherein the training dataset comprises data associated with treatments implemented using volumetric modulated arc therapy and intensity-modulated radiotherapy modalities; train the artificial intelligence model using the training dataset, such that the artificial intelligence model is configured to receive data associated with a new patient and generate a predicted dose map for the new patient indicating dosage received by one or more internal structures of the new patient; execute the artificial intelligence model for the new patient to receive the predicted dose map for the new patient; and output the predicted dose map for the new patient.

Outputting the predicted dose map may comprise displaying the predicted dose map on an electronic device.

Outputting the predicted dose map may comprise generating a fluence map based on the predicted dose map.

Outputting the predicted dose map comprises transmitting the predicted dose map to a plan optimizer, whereby the plan optimizer generates a treatment plan for the new patient using the predicted dose map.

The server may be further configured to compare the predicted dose map with at least one clinical goal.

The server may be further configured to generate a dose-volume histogram based on the predicted dose map.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of an artificial intelligence-based dose modeling system, according to an embodiment.

FIG. 2 illustrates a process flow diagram of an artificial intelligence-based dose modeling system, according to an embodiment.

FIG. 3A illustrates a visual depiction of patient and treatment data, according to an embodiment.

FIG. 3B illustrates a visual example of pre-processing performed in an artificial intelligence-based dose modeling system, according to an embodiment.

FIG. 4 illustrates non-limiting examples of dose maps and beam geometry, according to an embodiment.

FIG. 5A illustrates different components of an artificial intelligence model trained/implemented in an artificial intelligence-based dose modeling system, according to an embodiment.

FIG. 5B illustrates non-limiting examples of dose maps generated by an artificial intelligence-based dose modeling system, according to an embodiment.

FIG. 6 illustrates comparisons between different dose-volume histograms, according to an embodiment.

FIG. 7 illustrates a process flow diagram of an artificial intelligence-based dose modeling system, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

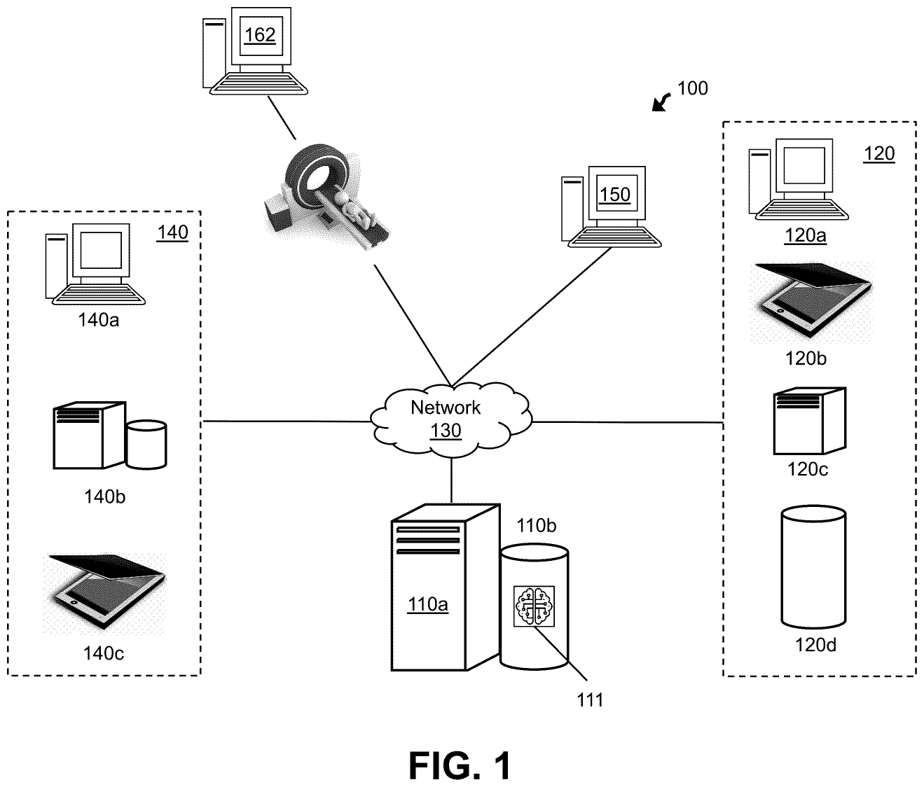

FIG. 1 illustrates components of a system 100 for an artificial intelligence-based dose modeling system, according to an embodiment. The system 100 may include an analytics server 110a, system database 110b, an AI model 111, electronic data sources 120a-d (collectively electronic data sources 120), end-user devices 140a-c (collectively end-user devices 140), an administrator computing device 150, and medical device 160, and medical device computer(s) 162. Various components depicted in FIG. 1 may belong to a radiotherapy clinic at which patients may receive radiotherapy treatment, in some cases via one or more radiotherapy machines located within the clinic (e.g., medical device 160).

The system 100 is not confined to the components described herein and may include additional or other components, not shown for brevity, which are to be considered within the scope of the embodiments described herein.

The above-mentioned components may be connected to each other through a network 130. Examples of the network 130 may include, but are not limited to, private or public local-area-networks (LAN), wireless LAN (WLAN) networks, metropolitan area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), and EDGE (Enhanced Data for Global Evolution) network.

The analytics server 110a may generate and display an electronic platform configured to use various AI model 111 (including artificial intelligence and/or machine learning models) for receiving patient information and outputting the results of execution of the AI model 111. The electronic platform may include graphical user interfaces (GUI) displayed on each electronic data source 120, the end-user devices 140, the medical device 160, and/or the administrator computing device 150. An example of the electronic platform generated and hosted by the analytics server 110a may be a web-based application or a website configured to be displayed on different electronic devices, such as mobile devices, tablets, personal computers, and the like.

The information displayed by the electronic platform can include, for example, input elements to receive data associated with a patient being treated, synchronize one or more sensors, and display results of predictions produced by the AI model 111. For instance, the analytics server 110a may execute the AI model 111 (e.g., machine learning models trained to generate predicted dose maps). The analytics server 110a may then display the results for a medical professional and/or directly revise one or more operational attributes of the medical device 160. In some embodiments, the medical device 160 can be a diagnostic imaging device or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various electronic data sources that contain, retrieve, and/or access data associated with a medical device 160, such as operational information associated with previously performed radiotherapy treatments (e.g., electronic log files or electronic configuration files), data associated with previously monitored patients or participants in a study to train the AI models discussed herein. For instance, the analytics server 110a may use the clinic computer 120a, medical professional device 120b, server 120c (associated with a physician and/or clinic), and database 120d (associated with the physician and/or the clinic) to retrieve/receive data associated with the medical device 160. The analytics server 110a may retrieve the data from the end-user devices 120, generate a training dataset, and train the AI models 111. The analytics server 110a may execute various algorithms to translate raw data received/retrieved from the electronic data sources 120 into machine-readable objects that can be stored and processed by other analytical processes as described herein.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, and server computer. In operation, various users may use end-user devices 140 to access the GUI operationally managed by the analytics server 110a. Specifically, the end-user devices 140 may include clinic computer 140a, clinic server 140b, and a medical professional device 140c. Even though referred to herein as “end-user” devices, these devices may not always be operated by end-users. For instance, the clinic server 140b may not be directly used by an end user. However, the results stored on the clinic server 140b may be used to populate various GUIs accessed by an end user via the medical professional device 140c.

The administrator computing device 150 may represent a computing device operated by a system administrator. The administrator computing device 150 may be configured to display radiotherapy treatment attributes generated by the analytics server 110a (e.g., various analytic metrics determined during training of one or more machine learning models and/or systems); monitor various models 111 utilized by the analytics server 110a, electronic data sources 120, and/or end-user devices 140; review feedback; and/or facilitate training or retraining (calibration) of the AI model 111 that are maintained by the analytics server 110a.

The medical device 160 may be a radiotherapy machine configured to implement a patient's radiotherapy treatment. The medical device 160 may also include an imaging device capable of emitting radiation such that the medical device 160 may perform imaging according to various methods to accurately image the internal structure of a patient. For instance, the medical device 160 may include a rotating system (e.g., a static or rotating multi-view system). A non-limiting example of a multi-view system may include stereo systems (e.g., two systems may be arranged orthogonally). The medical device 160 may also be in communication with a medical device computer 162 that is configured to display various GUIs discussed herein. For instance, the analytics server 110a may display the results predicted by the AI model 111 on the computing devices described herein.

The AI model 111 may be stored in the system database 110b. The AI model 111 may be trained using data received/retrieved data from the electronic data sources 120 and may be executed using data received from the end-user devices and/or the medical device 160. In some embodiments, the AI model 111 may reside within a data repository local or specific to a clinic. In various embodiments, the AI model 111 uses one or more deep learning engines to generate predicted dose maps. For instance, the analytics server 110a may transmit patient attributes from the sensor 163 and execute the AI model 111 accordingly.

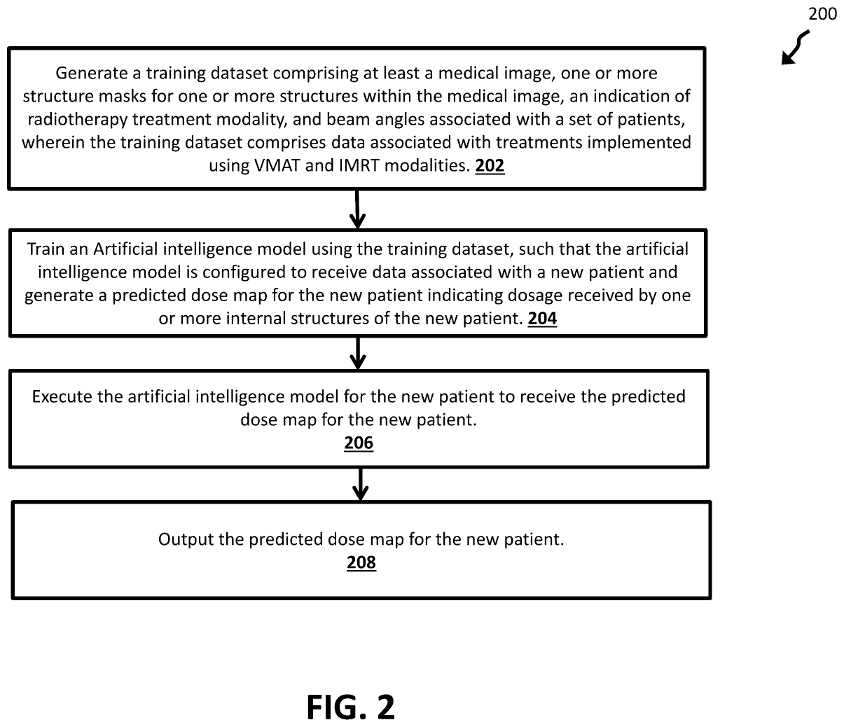

Referring to FIG. 2, depicted is an example data flow diagram 200 of a method performed by an artificial intelligence-based dose modeling system, according to an embodiment. The method 200 may include steps 202-208. However, other embodiments may include additional or alternative steps or may omit one or more steps altogether. The method 200 is described as being executed by a server, such as the analytics server described in FIG. 1. However, one or more steps of method 200 may be executed by any number of computing devices operating in the distributed computing system described in FIG. 1. For instance, one or more computing devices may locally perform part or all of the steps described in FIG. 2.

Radiotherapy treatment planning is a complex process that requires a multidisciplinary team, including oncologists, therapists, and physicists. Their role is to determine the optimal beam configurations and intensities for treating cancer patients. Contemporary radiotherapy treatments mainly fall into two categories: those that use static beams and those that use dynamic beams.

Intensity-modulated radiotherapy (IMRT) is a common method that employs static beams. In IMRT, multiple tailored yet fixed beam geometry are used. This method delivers radiation with high precision directly to the tumor, all while minimizing exposure to the surrounding healthy tissues. The beam's alignment takes into account the tumor's position and the nearby organs at risk (OARs). In contrast, Volumetric Modulated Arc Therapy (VMAT) uses dynamic beams. In VMAT treatments, the beam remains active while its treatment head moves in an arc trajectory.

Various noticeable differences exist between dose maps for static and dynamic beam radiotherapy plans. These variations arise due to the different energy delivery characteristics of the two methods. Furthermore, even when using the same planning approach, the configurations, such as beam geometry and isocenter, may differ from one patient to another. This may be due to the fact that different patients typically have unique tumor locations, shapes, anatomical structures, and other clinical parameters that necessitate a personalized approach.

An important aspect of knowledge-based planning may be a computer's ability to predict the dose distribution required for a patient. Using the method 200, a server (e.g., analytics server) may train an AI model, such that the AI model can learn patterns and commonalities among treatment attributes of a set of previously treated or test patients to train itself during its training phase. The AI model may then receive data associated with a new patient and generate a predicted dose map for the new patient. The AI model may predict a three-dimensional (3D) dose distribution for the new patient.

The AI model, as discussed herein, may be a dual-conditional generative adversarial net (DC-GAN model). The AI model may be trained and configured for precision heterogeneous 3D dose prediction, where the dual conditions may be dose modality and beam geometry (e.g., beam configurations). As discussed herein, IMRT and VMAT are major radiotherapy modalities used in the practice. However, IMRT and VMAT have clear distinctive patterns in terms of dose distribution maps. In some embodiments, a hybrid approach may be achieved where the two modalities are both used (e.g., used in combination). With the modality condition, the AI model can generate the desired dose with the accurate modality (e.g., IMRT, VMAT, or the hybrid approach). Given the beam geometry condition in the adversarial model, the AI model may predict a precision realistic dose map with given angles.

At step 202, the analytics server may generate a training dataset comprising at least a medical image, one or more structure masks for one or more structures within the medical image, an indication of radiotherapy treatment modality, and beam geometry associated with a set of patients, wherein the training dataset comprises data associated with treatments implemented using VMAT and IMRT modalities. The analytics server may retrieve a radiotherapy planning treatment (RTTP) for the set of patients and generate the training dataset accordingly.

The analytics server may retrieve and aggregate treatment data associated with a set of patients. As used herein, the set of patients may refer to a set of previously treated patients and/or test patients, such as patients who have participated in a trial for the purposes of gathering information. The patient data may be processed, such that a training dataset is generated. The patient data retrieved may include treatment data/patient data for a set of patients that includes treatments administered via both IMRT and VMAT modalities. In some embodiments, the training dataset may include patient data for one or more patients who received a hybrid treatment (e.g., a combination of VMAT and IMRT).

A non-limiting example of the patient data collected may be medical images of the patients. As used herein a medical image may refer to any medical image depicting one or more of the patient's internal structures, such as the PTV/GTV and OARs. A non-limiting example of a medical image may be a CT scan/image. However, the methods and systems discussed herein apply to all medical images (e.g., MRI) and are not limited to CT scans/images.

Another non-limiting example of the patient data may include a dose map or a reference dose map for each patient. As used herein, a dose map or a dose distribution map may represent the spatial distribution of radiation dose delivered to a target volume (e.g., PTV) and surrounding tissues within a patient (OARs). A dose map may be represented/displayed as a two-dimensional (2D) or three-dimensional (3D) representation, using a combination of colors or contour lines to indicate different dose levels.

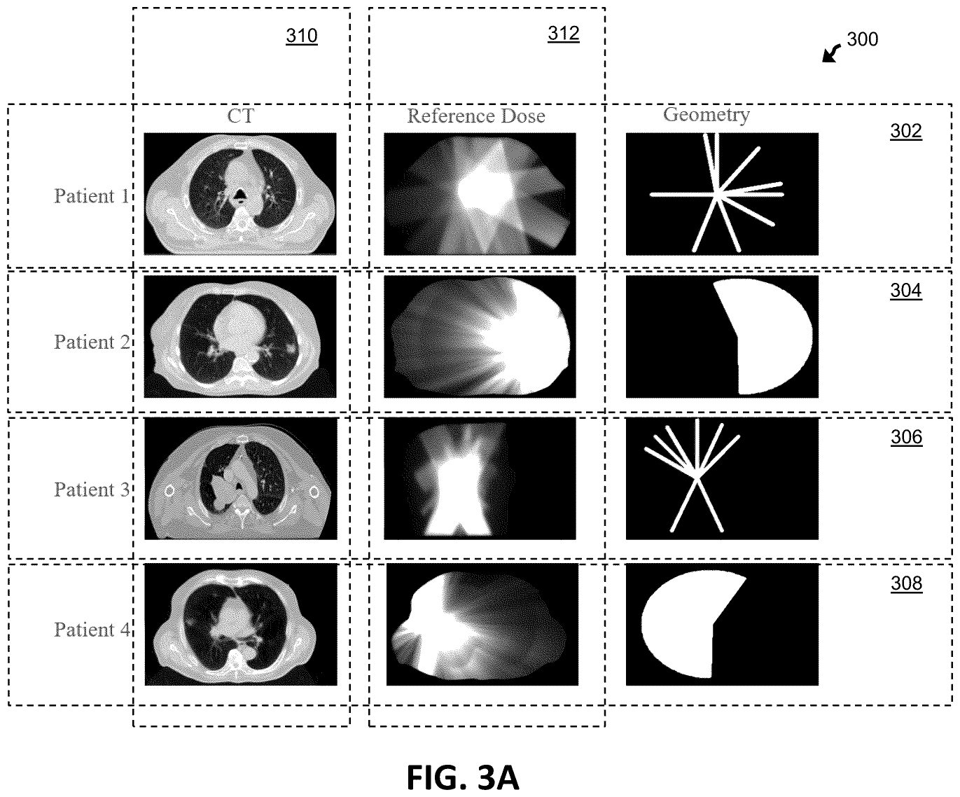

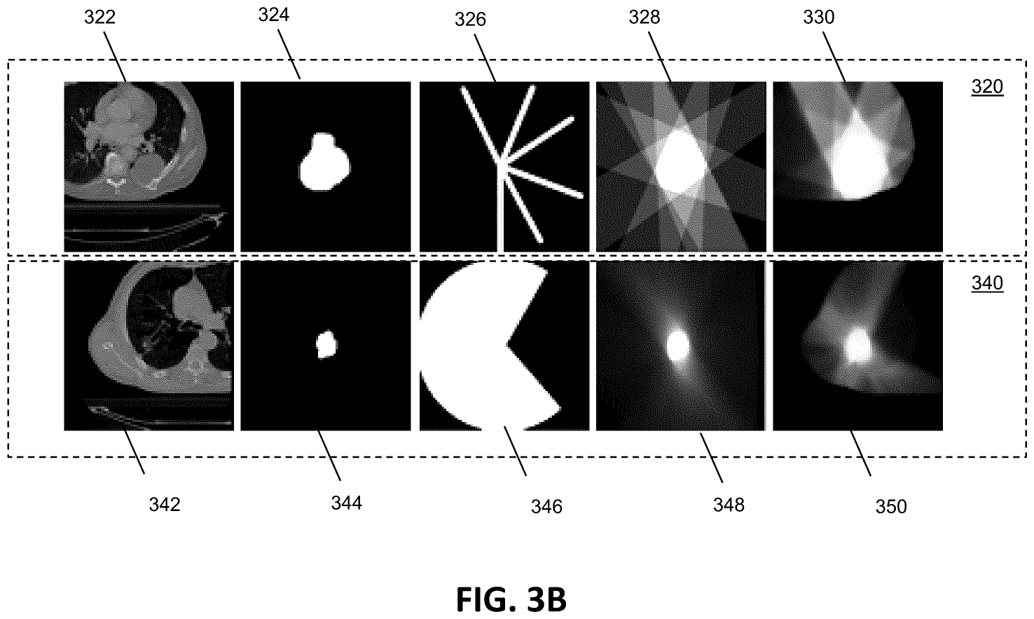

Before training the AI model, the analytics server may generate a training dataset using approved dose plans for the patients (e.g., previously treated patients and/or test patients). In some embodiments, the analytics server may retrieve and aggregate medical images (CT scans) of a set of patients, along with an approved reference dose map and beam geometry for each respective patient's treatment. Referring now to FIG. 3A, a visual depiction of the training dataset 300 is provided, according to an embodiment. The training dataset 300 may depict only certain aspects of the overall training dataset used to train the AI model. Therefore, the training dataset 300 does not limit the content of the overall training dataset that can be used to train the AI model. As described herein, various other information can be added to the training dataset before the training dataset is used to train the AI model.

FIG. 3A illustrates the high heterogeneity of approved dose plans from different patients. In some embodiments, it may be inefficient to train the AI model from all the reference doses without any additional/complementary information regarding the planning modality. Specifically, it may be technically challenging to attempt to train an AI model using the CT scans depicted within a column 310 along with approved reference dosages depicted within a column 312. This is because different modalities use beam geometry that are different in nature. In order to rectify this technical shortcoming, a beam geometry representation or mask may be generated. For instance, patient 1 (row 302) has received lung cancer treatment using IMRT. As depicted, the beam geometry for patient 1 looks entirely different than the beam geometry for patient 2 (row 304) who has also received lung cancer treatment, but via a different modality (using VMAT). Patients 3 and 4 have also received treatment in comparable anatomical regions using different modalities. As a result, their respective beam geometry is different.

The beam geometry can sometimes be decided by the plan optimizing software and/or a medical professional. For instance, the beam geometry may be fed into the optimizer, such that the optimizer can generate an RTTP accordingly (e.g., optimize the MLC and Mu values). Therefore, in some embodiments, the beam geometry may be included within the RTTP.

The analytics server may use various pre-processing protocols to improve the training dataset. In some embodiments, the analytics server may leverage radiotherapy geometry by generating spatial matrices (sometimes referred to as plates). As depicted in FIG. 3B, the “angle plates” may be binary masks representing angles (resembling sectors for dynamic beam plates due to their span over a range of angles). The beam plates may be generated based on angles and PTV masks, incorporating and implementing known geometry information.

FIG. 3B illustrates two samples. The sample 320 may be beam-static and sample 340 may be beam-dynamic. The depicted angle plates and beam plates may be created using the CT images (322 and 342) and PTV/OAR masks (324 and 344), which are sometimes referred to collectively as the source data. Using the source data, a pre-processing protocol can generate the angle plate and beam plates (326-328 and 346-348) while optionally using the reference doses 330 and 350.

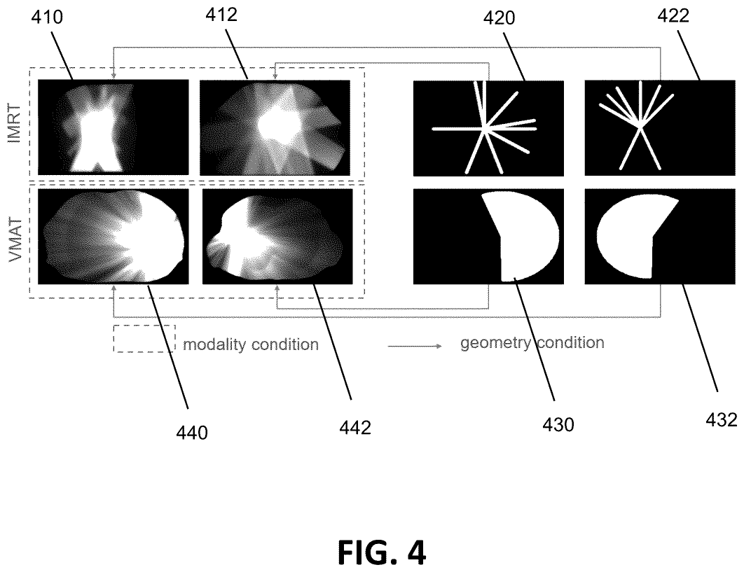

In some embodiments, the analytics server may identify/generate conditions and corresponding angle maps or beam geometry using the dose maps. For instance, as shown in FIG. 4, the first level condition may be the modality of the treatment, which can inform the AI model of the type of dose planning to be predicted. The second level condition may be the beam geometry of the treatment, which provides some information about the beam geometry configuration. As depicted, given the dose maps 410 and 412, the analytics server may determine the corresponding treatment modality (IMRT) and generate the beam geometries 420 and 422. Similarly, given dose maps 440 and 442, the analytics server may determine the modality to be VMAT and generate the beam geometry 430 and 432.

Another example of the patient data within the training dataset may include structure masks. As used herein, a mask may refer to a contour of one or more structures (PTVs and OARs) depicted within the medical images.

At step 204, the analytics server may train an artificial intelligence model using the training dataset, such that the artificial intelligence model is configured to receive data associated with a new patient and generate a predicted dose map for the new patient indicating dosage received by one or more internal structures of the new patient. Using the training dataset discussed herein, the AI model may be trained, such that it can provide/predict dual conditions (e.g., modality condition and geometry condition) for the AI model to learn the desired dose patterns.

The AI model discussed herein (the DC-GAN model) may be a deep learning model that can generate multi-nodal dose maps when given the modality code (e.g., an indication of the modality of treatment). The AI model may also be a deep learning model that generates precision dose utilizing beam geometry from heterogeneous configurations. The AI model may use a dual-conditional mechanism to handle various challenges with multiple levels of conditions. In addition, given the challenge of limited data in the medical domain, various loss functions may be used to enforce the conditions highlighted in generated dose maps. This allows the AI model to require a smaller training dataset. Accordingly, a cross-entropy loss regularization may be used to push the generated dose to belong to the desired modality. Moreover, a topology-preserving regularization may be used to obtain the desired geometry.

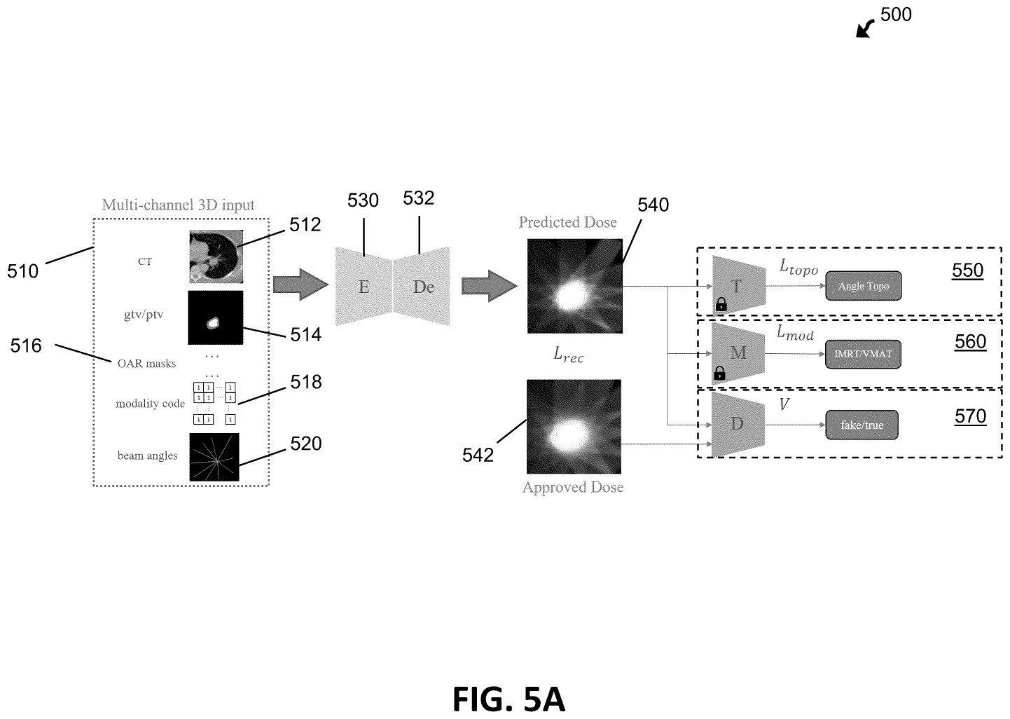

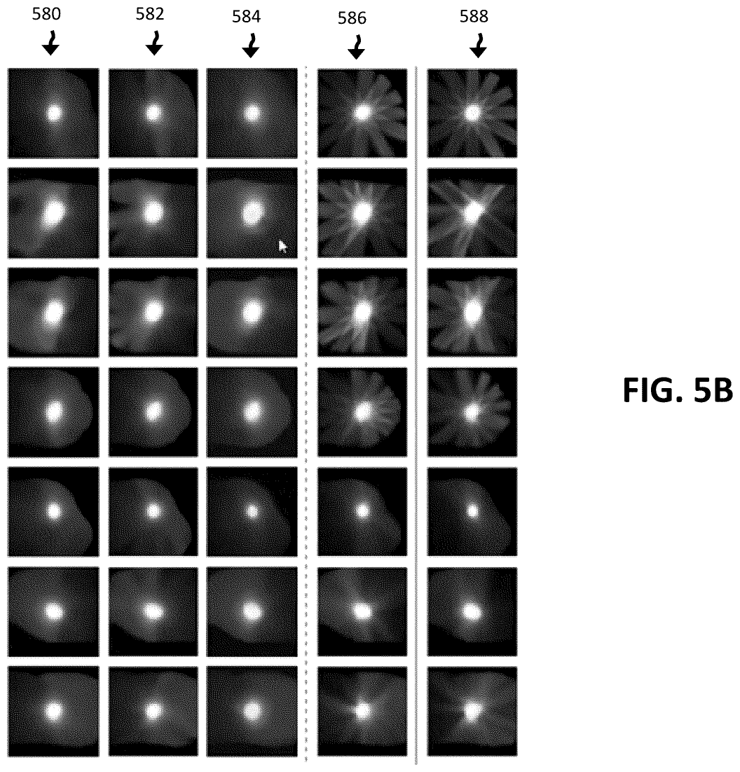

FIG. 5A illustrates different components of an artificial intelligence model trained/implemented in an artificial intelligence-based dose modeling system, according to an embodiment. The AI model 500 (and its various modules) is illustrated in FIG. 5A, in accordance with an embodiment. As depicted, the AI model 500 ingests multi-channel 3D inputs (the training dataset) to train itself. The input (sometimes referred to as the input x) may consist of multiple 3D tensors including one or more medical images (e.g., CT image 512) and their corresponding organ identifications (e.g., GTV/PTV masks 514 and other PTV identification data), the corresponding OAR masks 516. As used herein a mask is an indication/contouring of a target structure. For instance, an OAR mask may refer to an outline of an OAR within a medical image, such as CT image 512.

The multi-channel 3D input may also consist of a modality tensor 518, which may include binary data indicating a modality of the corresponding data. For instance, the modality tensor 518 may be a 3D duplication of the modality code (IMRT: 0 and VMAT 1). In embodiments where a hybrid approach is taken, a new code may be assigned to the hybrid modality. In some embodiments, the modality tensor 518 may match the dimensionality of the CT image 512, such that the underlying data can be concatenated together.

The multi-channel 3D input may also consist of a beam-angle tensor 520. As used herein, the beam-angle tensor 520 may refer to a tensor that is created from gantry angles. In a non-limiting embodiment, the gantry angles may be included within an RTTP.

The AI model 500 may train itself using the multi-channel 3D input, such that the AI model's output is predicted dose map with desired modality and beam geometry/angles.

The AI model 500 may include five modules: the encoder (E) 530, the decoder (De) 532, topo-net (T) 550, mod-net (M) 560, and the discriminator (D) 570. In some embodiments, the encoder 530 may extract the abstractive features from the multi-channel 3D input. The encoder 530 may consist of various convolutional neural network layers. The decoder 532 may be used to reconstruct the dose map from abstractive features. Therefore, the decoder 532 may ingest the results encoded/extracted by the encoder 530. Accordingly, the AI model's generator may be defined as E+De.

The topo-net (T) 550 and mod-net (M) 560 may be pre-trained using approved dose maps, such as dose maps that belong to previously treated patients and/or test patients where the dose map has been reviewed and approved by a medical professional and/or a protocol. The approved dose maps can be used as de facto ground truth to train the AI model 500. Accordingly, the topo-net 550 and the mod-net 560 may be used to extract topological features and modality features from the dose maps predicted by the AI model 500 respectively. The topo-net 550 may ensure that the topological attributes of the predicted dose map 540 match the corresponding attributes of 542.

The mod-net 560 may be a classification model that classifies based on whether the predicted data corresponds to VMAT or IMRT (or hybrid). Therefore, the mod-net 560 may ensure that the predicted dose has the same classification as the approved dose map 542.

Finally, the discriminator (D) 570 may be used to distinguish between the predicted dose maps 540 (fake or machine-generated) from the approved dose maps 542 (e.g., real or actual dose maps). When trained, the discriminator 570 can be used for adversarial training, such that the AI model 500 can generate realistic samples.

The AI model may use various loss functions during training. The AI model 500 may use the loss functions for the precision heterogeneous dose prediction.

In a non-limiting example, the AI model 500 may use a reconstruction loss L_{rec} during training. The reconstruction loss L_{rec} may be utilized to minimize the difference between predicted dose (ŷ) and approved dose (y) in 3D matrix space and Dose Volume Histogram (DVH) space, which may be defined as follows:

L_{rec}=||y−ŷ||+||DVH(y)−DVH(ŷ)||, ŷ=De(E(x)).

In another non-limiting example, the AI model 500 may use a topology loss Lₜₒₚₒ during training. The topology loss Lₜₒₚₒ may be used to extract topology features to match the beam geometry information x_{T}, which is defined as:

Lₜₒₚₒ=||T(ŷ)−x_{T}||.

In another non-limiting example, the AI model 500 may use a modality loss L_{mod} during training. The modality loss may be used to ensure that the predicted dose has the desired modality x_{M}, which is defined as:

L_{mod}=−x_{M}logM(ŷ)−(1−x_{M})log(1−M(ŷ))

In another non-limiting example, the AI model 500 may use a discriminator loss L_{dis} during training. As discussed herein, the loss function indicates whether an image is machine-generated or real.

In some embodiments, L_{rec}, Lₜₒₚₒ, and L_{mod} may be only used for optimizing the generator (G={E, De}).

The AI model 500 may use a generative adversarial network paradigm to generate the predicted dose map 540. Using GANs, the AI model 500 may generate more realistic outputs. GAN's conditional variations may enable the imposition of additional limitations and constraints, which can be advantageous for dose map generation. A Conditional GAN may be characterized by a min/max game between the discriminator and a generator, with the following loss function:

V(D, G)= [symbol] _{x˜p} [symbol] [log D(x|y)]+ [symbol] _{x˜p} [symbol] _{(z)}[log(1−D(G(z|y)))],

where the generator may create outputs based on a given condition y and random noise z.

Moreover, the GAN loss may be optimized for training both the discriminator 570 and generator as shown below:

$${min}_{\{ E , D e \}} {max}_{D} V ( E , De , D ) = {E}_{x , y \sim p ( x , y )} [ log D ( y ) + log ( 1 - D ( D e ( E ( x ) ) ) ]$$

The AI model 500 may be trained to predict dose maps, even under challenging heterogeneous configurations. That is, the AI model 500 may be configured/trained to predict dose maps by ingesting a desired dose modality and beam geometry/angles. The AI model 500 may also predict dose-volume histograms (DVHs) for one or more target structures, such as the PTV and OARs.

The AI model 500 may be trained using the methods and techniques discussed herein, such that the predicted results depict dose maps that visually resemble the reference dose maps. As a result, the predicted/generated dose maps for a new patient may also resemble a “real” dose map. Referring now to FIG. 5B, the dose maps predicted/generated using the AI model 500 are compared to other AI approaches. As depicted, the dose maps within the column 586 (generated by the AI model 500) have similar visual characteristics when compared to the reference dose maps (column 588) when compared against dose maps within the rows 580, 582, and 582 predicted using Dilated-Residual U-Net Deep Learning Network (DRUNet), Compact Unsupervised Network (CUNet), and Swin Transformers respectively.

Using the loss functions discussed herein may allow for training the AI model 500 using fewer samples and a smaller training dataset compared to tasks in computer vision domain.

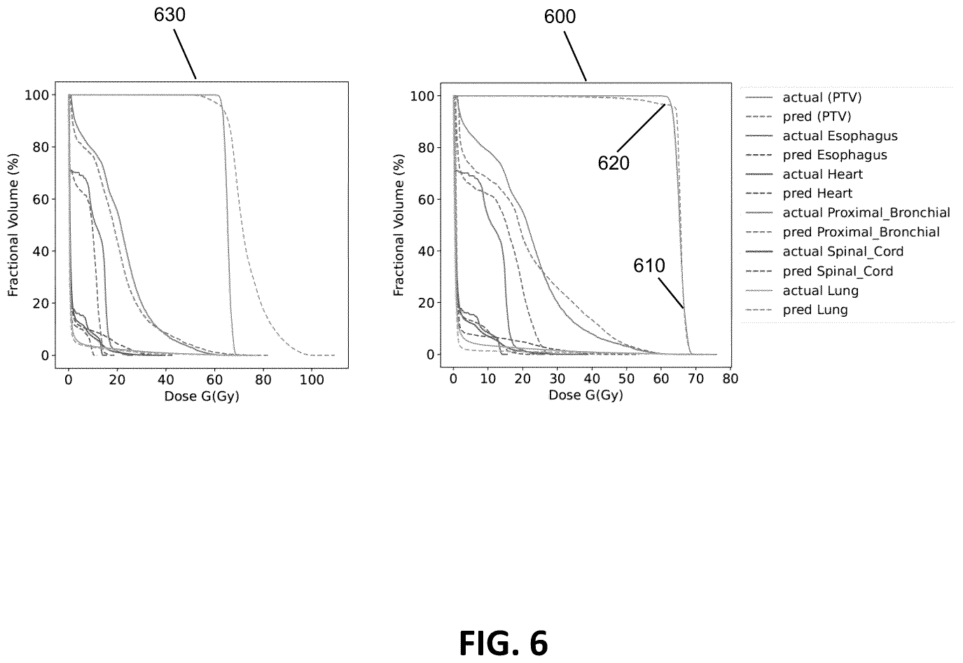

Referring back to FIG. 5A, the AI model 500 may also be validated using known data. For instance, a generated DVH (predicted by the AI model 500) may be compared against actual DVHs identified for previously treated patients. Referring now to FIG. 6, DVH 600 illustrates the DVH comparison between actual and predicted doses (predicted by the AI model discussed herein). The dashed lines may represent doses computed from the predicted dose map and solid lines may represent the dose calculated using the approved/actual dose. For instance, the line 610 may represent the actual dose of the PTV and the line 620 may represent dosage, as predicted using the AI model (or calculated using a dose map predicted by the AI model). As depicted, the values represented by the lines 610 and 620 indicate that the predicted values are accurate (e.g., within tolerable distance).

Similarly, the performance of the AI model can be compared with DVHs, such as the DVH 630, which have been predicted using other AI models, such as conventional AI models, or generated via other conventional methodologies. As illustrated the AI model trained using the techniques discussed herein predicts doses for different target structures that are much closer to actual values.

Referring back to FIG. 5A, when trained, the AI model 500 may be configured to receive patient data for a new patient and predict a 3D dose map for the new patient. For instance, the AI model 500 may predict the predicted dose map 540. Because the AI model 500 is trained using a training dataset that includes both modalities, the AI model 500 may predict data associated with both modalities. Therefore, unlike conventional approaches where different modalities were analyzed by separate models, the AI model 500 can predict dose maps regardless of the treatment modality.

Referring back to FIG. 2, at step 206, the analytics server may execute the AI model for a new patient to receive a predicted dose map for the new patient. The analytics server may receive patient data and treatment objectives and goals for a new patient. For instance, the analytics server may retrieve medical images(s) of the new patient along with masks and contours of PTV and OARs for the medical images. The analytics server may also receive clinical goals for the patient's treatment. In some embodiments, the analytics server may also receive a desired modality for the patient's treatment. The analytics server may execute the AI model (trained via the techniques discussed herein) and generate a predicted dose map for the new patients.

At step 208, the predicted dose map may be outputted by the analytics server. The predicted dose map may then be transmitted to one or more downstream software applications and/or displayed on a computing device. For instance, the predicted dose map for the new patient may be displayed for a treating physician or a clinician, such that the prediction can be used to make ultimate decisions regarding the new patient's treatment.

In another example, the predicted dose map can be used to check the quality of an RTTP for a new patient (e.g., before the treatment is implemented). Using the predicted dose map, a medical professional can compare the dosage received by different structures against various rules and thresholds to ensure that no structure (e.g., OAR) is receiving more dosage than desired. Moreover, DVH can be generated using the predicted dose maps, such that comparing dosages to be administered can be visually inspected.

In another example, the predicted dose map can be used to generate a fluence map. As used herein, a fluence map may illustrate the distribution of the number of radiation particles (e.g., photons or electrons) that pass through a given area. That is, a fluence map illustrates how beams of radiation are distributed spatially. The analytics server and/or a downstream software application may execute various protocols to generate a fluence map using the output of the AI model when trained using the methods and systems discussed herein.

In some embodiments, the AI model may be used to predict a dose map for a particular treatment plan and the doses predicted by the AI model may be compared with various clinical goals. For instance, the analytics server may compare the doses (predicted by the AI model) with various clinical goals (e.g., thresholds implemented by a medical professional). If a dose is higher or lower than expected, the analytics server may then transmit a notification to the medical professional.

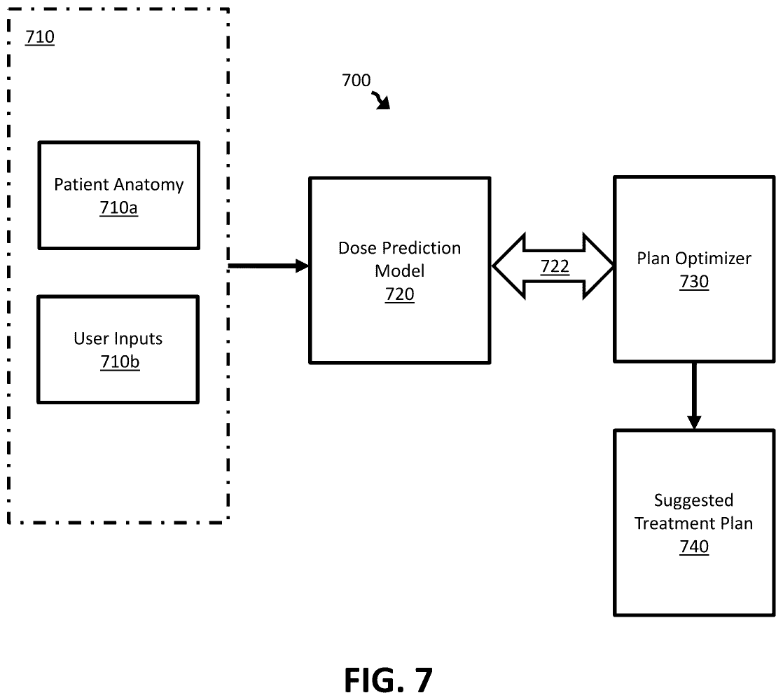

The AI model discussed herein can also be used within a pipeline of generating a radiotherapy treatment plan (RTTP). Referring now to FIG. 7, a non-limiting visual example of a workflow utilizing the methods and systems described herein is illustrated. In this non-limiting example 700, the analytics server provides dose predictions to a plan optimizer 730 to generate a suggested RTTP that is optimized for a patient and their treatment. The analytics server may first collect patient data 710. The patient data may include patient anatomy data 710a (e.g., medical images, PTVs, OARs), user inputs 710b (clinical objectives or rules received via a user interface from a treating oncologist, such as tumor data, PTV identification, and the like). The patient data 710 may also include a desired modality, though this input may not be required. In general, the patient data 710 may include any information needed to generate an RTTP.

In some configurations, the analytics server may access a patient's internal/external file and retrieve/extract the needed patient data 710. The analytics server may then execute a dose prediction AI model 720 to generate a dose map using the patient data 710. The dose prediction AI model 720 may be an AI model trained/implemented using the methods and systems discussed herein, such as the AI model 500 described in FIG. 5. The results generated via the dose prediction AI model 720 may be ingested by the plan optimizer 730. The plan optimizer 730 may be a treatment planning and/or monitoring software solution. The plan optimizer 730 may analyze various factors associated with the patient and the patient's treatment to generate and optimize an RTTP for the patient (e.g., field geometry, treatment modality, and radiation parameters needed to treat the patient).

One of the factors considered by the plan optimizer 730 may be the dose distributions and predictions predicted by the dose prediction AI model 720.

The plan optimizer 730 may iteratively revise the patient's RTTP where the plan optimizer 730 iteratively revises different attributes of the RTTP. In some configurations, the plan optimizer 730 may transmit new treatment plan data back to the dose prediction AI model 720, whereby the dose prediction AI model 720 can recalculate/re-predict data based on the revised treatment data generated by the plan optimizer (iteration 722).

In some embodiments, the dose prediction AI model 720 may receive new data corresponding to a new RTTP from the plan optimizer 730 and generate a predicted dose map accordingly where the predicted dose map can be used to determine the quality of the new RTTP. Therefore, the methods and systems discussed herein can be used to automatically compare dosages generated by an RTTP.

When the plan optimizer completes the patient's RTTP, the plan optimizer 730 may transmit the suggested treatment plan 740 to one or more electronic devices where a user (e.g., clinician) can review the suggested plan. For instance, the suggested treatment plan 740 may be displayed on a computer of a clinic where a radiation therapy technician or a treating oncologist can review the treatment plan.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate the transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
generating, by a processor, a training dataset comprising at least a medical image, one or more structure masks for one or more structures within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities;
training, by the processor, an artificial intelligence model using the training dataset, such that the artificial intelligence model is configured to receive data associated with a new patient and generate a predicted dose map for the new patient indicating dosage received by one or more internal structures of the new patient;
executing, by the processor, the artificial intelligence model for the new patient to receive the predicted dose map for the new patient; and
outputting, by the processor, the predicted dose map for the new patient.

2. The method of claim 1, wherein outputting the predicted dose map comprises:
displaying, by the processor, the predicted dose map on an electronic device.

3. The method of claim 1, wherein outputting the predicted dose map comprises:
generating, by the processor, a fluence map based on the predicted dose map.

4. The method of claim 1, wherein outputting the predicted dose map comprises:
transmitting, by the processor, the predicted dose map to a plan optimizer, whereby the plan optimizer generates a treatment plan for the new patient using the predicted dose map.

5. The method of claim 1, further comprising:
comparing, by the processor, the predicted dose map with at least one clinical goal.

6. The method of claim 1, further comprising:
generating, by the processor, a dose-volume histogram based on the predicted dose map.

7. The method of claim 1, wherein the artificial intelligence model receives a modality indicator from the processor before generating the predicted dose map for the new patient.

8. A computer system comprising:
a server comprising a processor and a non-transitory computer-readable medium containing instructions that when executed by the processor causes the processor to:
generate a training dataset comprising at least a medical image, one or more structure masks for one or more structures within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities;
train an artificial intelligence model using the training dataset, such that the artificial intelligence model is configured to receive data associated with a new patient and generate a predicted dose map for the new patient indicating dosage received by one or more internal structures of the new patient;
execute the artificial intelligence model for the new patient to receive the predicted dose map for the new patient; and
output the predicted dose map for the new patient.

9. The computer system of claim 8, wherein outputting the predicted dose map comprises displaying the predicted dose map on an electronic device.

10. The computer system of claim 8, wherein outputting the predicted dose map comprises generating a fluence map based on the predicted dose map.

11. The computer system of claim 8, wherein outputting the predicted dose map comprises transmitting the predicted dose map to a plan optimizer, whereby the plan optimizer generates a treatment plan for the new patient using the predicted dose map.

12. The computer system of claim 8, wherein the instructions further cause the processor to:
compare the predicted dose map with at least one clinical goal.

13. The computer system of claim 8, wherein the instructions further cause the processor to:
generate a dose-volume histogram based on the predicted dose map.

14. The computer system of claim 8, wherein the artificial intelligence model receives a modality indicator from the processor before generating the predicted dose map for the new patient.

15. A computer system comprising:
an artificial intelligence model; and
a server in communication with the artificial intelligence model, the server configured to:
generate a training dataset comprising at least a medical image, one or more structure masks for one or more structures within the medical image, an indication of radiotherapy treatment modality, and beam geometry associated with a set of patients, wherein the training dataset comprises data associated with treatments implemented using at least one of volumetric modulated arc therapy or intensity-modulated radiotherapy modalities;
train the artificial intelligence model using the training dataset, such that the artificial intelligence model is configured to receive data associated with a new patient and generate a predicted dose map for the new patient indicating dosage received by one or more internal structures of the new patient;
execute the artificial intelligence model for the new patient to receive the predicted dose map for the new patient; and
output the predicted dose map for the new patient.

16. The computer system of claim 15, wherein outputting the predicted dose map comprises displaying the predicted dose map on an electronic device.

17. The computer system of claim 15, wherein outputting the predicted dose map comprises generating a fluence map based on the predicted dose map.

18. The computer system of claim 15, wherein outputting the predicted dose map comprises transmitting the predicted dose map to a plan optimizer, whereby the plan optimizer generates a treatment plan for the new patient using the predicted dose map.

19. The computer system of claim 15, wherein the server is further configured to:
compare the predicted dose map with at least one clinical goal.

20. The computer system of claim 15, wherein the server is further configured to:
generate a dose-volume histogram based on the predicted dose map.
